(19) United States
(12) Patent Application Publication
(10) Pub. No.: US 2006/0271130 A1
(43) Pub. Date: Nov. 30, 2006

(54) Transmission device for introducing light into an ear

(76) Inventors: Andreas Mayer-Gindner, Stutensee (DE); Winfred Granget, Iapringan (DE)

Correspondence Address: DENNISON, SCHULTZ & MACDONALD, 1727 KING STREET, SUITE 105, ALEXANDRIA, VA, 22314, US

(21) Appl. No.: 10/524,774

(22) PCT Filed: Aug. 14, 2003
(86) PCT No.: PCT/DE03/02732

§ 371 Date: May 26, 2006

(30) Foreign Application Priority Data
Aug. 16, 2002 (DE) 102 37 620.4

(51) Int. Cl. A61N 5/06

(52) U.S. Cl. 607/88

(57) ABSTRACT

A transmission device for introducing optical radiation into the ear, including an optical wave guide which connects a light source to an ear holder, which includes a first transition piece, preferably elbow-shaped, in which the end area of the optical wave guide is maintained, and a retaining bar which can be introduced into the transition piece and bent in such a manner that the outlet of the optical wave guide can be positioned on the ear. A second transition piece, which is conically tapered on the inside and which is at least partially used as a focus bushing for the laser beam is provided in order to inject optical, preferably laser, radiation into the optical wave guide. The optical wave guide is positioned in the second transition piece such that the focus area of the laser beam produced in the focus bushing is disposed directly in front of the inlet of the optical wave guide.

### PRIOR ART

The use of a light source, for example a laser light source having low power, for treating discomforts in the region of the ear is for example known from document DE 94 02 306.9 for the treatment of Tinnitus. Document DE 100 49 068 A1 and document DE 199 47 678 C1 also refer to corresponding devices.

The basic structure of all these devices is similar regarding a light source, which is used for providing the emission and the introduction of the radiation into the ear region, a suitable holder at the ear and a wave guide, in particular an optical wave guide, which is suitable for conducting the radiation in the frequency range used.

The duration of the treatment for corresponding appliances is about 15 to 60 minutes when using a low level laser; accordingly, it has to be ensured that minimum comfort requirements are provided by means of the configuration of the holder when the devices are carried by a patient.

Regarding medical importance, the intensity of the radiation used and the position of exposure are essential, which ensures an effect on the cochlea; both parameters determine essentially the therapeutic effectivity of the radiation, and are as well critical for avoiding damages to the health which might occur due to too strong and/or incorrectly positioned radiation exposure.

The solution of DE 94 02 306 lacks comfort due to the use of an ear phone commonly available on the market, because of the remarkable weight of the ear phone; the radiation is not directed to the auditory canal, but to the region behind the ear on the cranium. The medical effect remains, therefore, doubtful, because the absorption of visible radiation by the bones is high.

DE 100 49 068 A1 deals with the positioning and exposure of incoherent radiation into the ear region and comprises a detection device for the intensity of the light for avoiding damages to the health, which supervises an individual value and controls the light emission device by means of a feedback circuit. This device is, accordingly, only usable as a complete unit.

The positioning device is to be introduced into the auditory canal of the patient, such that a radiation acts via the outer auditory canal and the ear drum on predetermined regions of the middle ear and/or the inner ear. The determination of these regions depending on the frequency range of the bothersome tone or sound is medically unclear and troublesome.

DE 199 47 678 uses a low level laser as an emission source with a protective element between the radiation source and the optical wave guide for avoiding laser emissions without connected optical wave guides. There is no disclosure regarding the construction of the holder.

### SUMMARY OF THE INVENTION

It is an object of the invention to provide a device which allows the transmission of light to the ear of the patient in a reliable manner, which is easy to handle and individually adjustable.

This object is solved by the invention by means of the features of the characterizing clause of claim 1.

The basic idea of the invention is the two-piece construction of an ear holder having a first transition piece which is formed such that it emits the radiation used in the direction of the auditory canal considering the orientation of the optical wave guide, and a retaining bar which is fixable to the first transition piece and which causes the individual fine adjustment and positioning of the transition piece.

According to a preferred embodiment a second transition piece is used for introducing the radiation into the optical wave guide, which in turn is a two-piece construction, namely a first piece for connecting to the radiation source, in particular a laser, and a second piece, a pressing tip, which is fixedly connected to the inlet end of the optical wave guide and introducable into the first piece.

Further embodiments are disclosed in the remaining dependent claims.

### SHORT DESCRIPTION OF THE FIGURES

A preferred embodiment of the transmission device according to the invention is now described in more detail based on the figures, in which:

FIG. 1: shows a perspective overall view of the transmission device;

FIG. 2: shows a top view of the ear holder comprising a first transition piece and a retaining bar,

FIG. 3: is a schematic sectional view of the transmission device according to FIG. 1,

FIG. 4: shows a section of a part of the construction of the second transition piece, and

FIG. 5: is a schematic view of the assembly of an exposure device having the transmission device according to the invention.

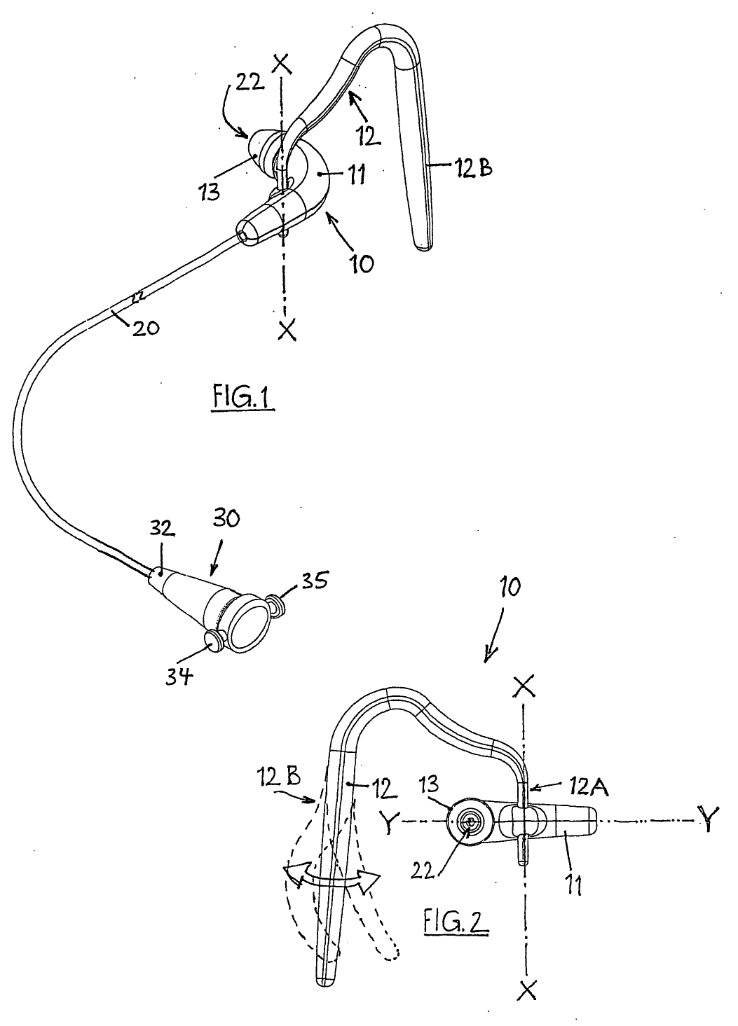

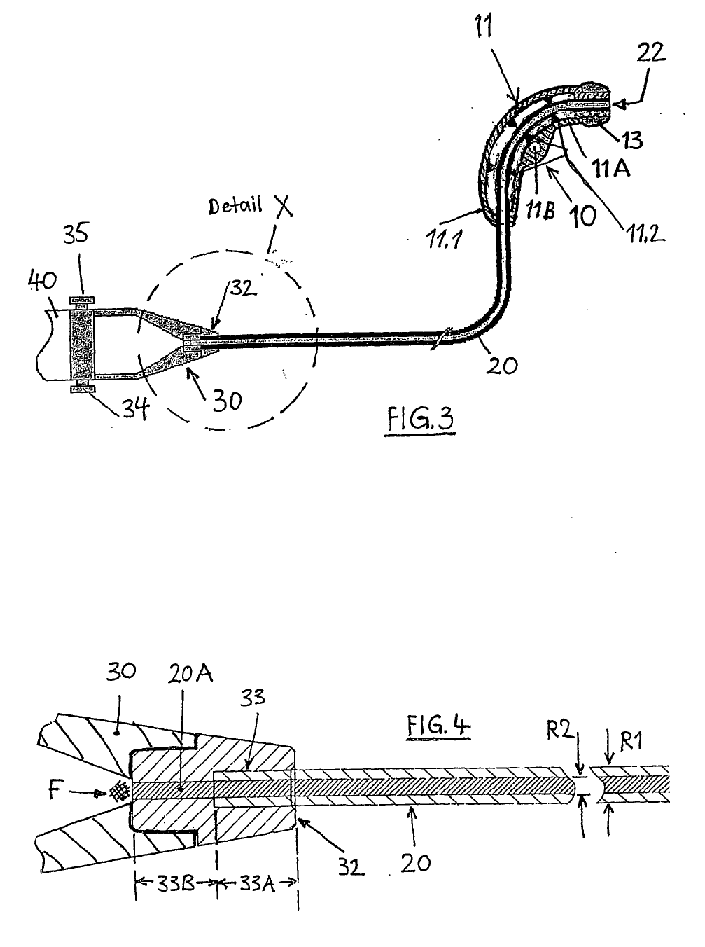

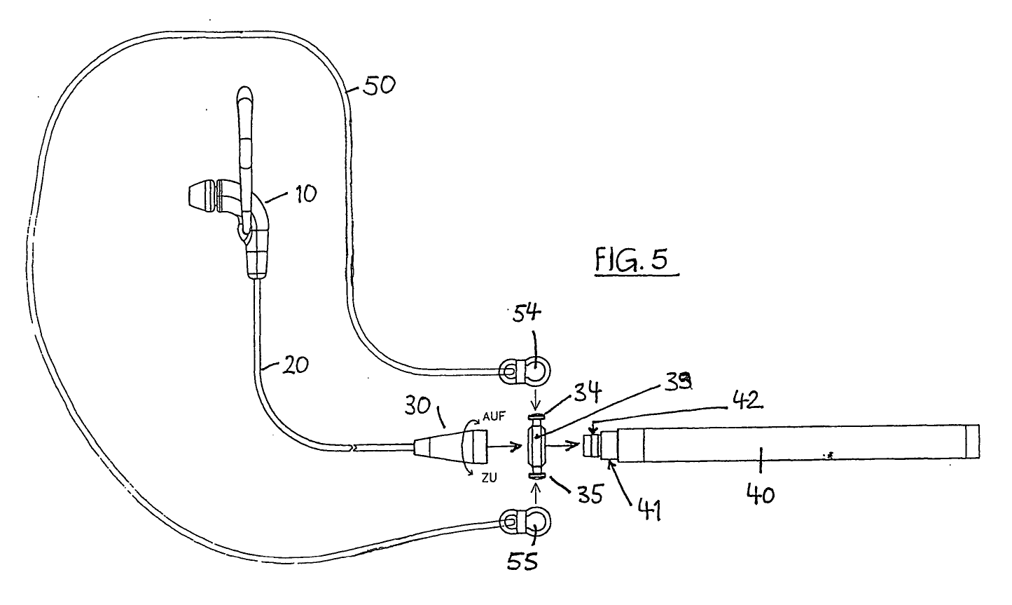

### DESCRIPTION OF THE PREFERRED EMBODIMENT

The following is based on the fact that the radiation of a low energetic laser (low level laser) is used as optical radiation; however, the use of any other radiation source of another frequency or another frequency spectrum is also possible.

The transmission device comprises essentially three parts: an optical wave guide 20, an ear holder 10, in which the end of the optical wave guide 20 of the ear side is accommodated, and a transition piece 30, in which the end of the optical wave guide 20 of the laser side is accommodated, for connecting to a suitable low level laser 40.

The ear holder 10 comprises a first transition piece 11, which is preferably embodied in a elbow shaped manner, for causing a re-direction of the optical wave guide 20, which usually is in a vertical plane, into the essentially horizontally running auditory canal of the ear, into which the laser radiation is to be introduced.

The optical wave guide 20 is thin (at maximum 1000 μm) and, accordingly, flexible, such that it can be introduced into the transition piece 11 or inserted into the transition piece 11 using a bending radius of 2 to 5 cm. This enables a very compact construction of the transition piece 11, which in turn reduces the lever formed, such that the moment caused by a part of the self-weight of the optical wave guide 20 around the support point of the transition piece 11 at the ear is kept minimum.

The transition piece 11 comprises two channel shaped half shells one of which (11.1) is shown in section from above in FIG. 3. In this half shell several ridges 11.2 in transverse direction are provided into which the optical wave guide 20 is inserted. Subsequently, the second half shell is attached and welded by means of ultrasonic welding, such that the wedge-shaped ridges 11.3 are pressed into the jacket of the optical wave guide 20 which is thereby locked.

The end portion 11A of the first transition piece 11 directed to the ear is covered by a replaceable ear piece 13, formed for example of a soft synthetic material, which forms the contact to the ear and is replaceable for hygienic reasons. Different sizes of the ear piece 13 serve for the adjustment to the ears of children and adults.

A through hole 11B is formed perpendicularly to the plane formed by the first elbow-shaped transition piece 11 through the elbow-shaped transition piece 11 (axis X-X), into which the first end portion 12A of a retaining bar 12 is rotatably inserted. The retaining bar 12 has a substantially U-shaped configuration, wherein its second end portion 12B is elastically deformable, especially bendable, such that it is adjustable to the shape of the cochlea, such that a secure fixing of the ear holder is ensured at the ear of the patient in combination with the ear piece 13.

The ear holder 10 shown is usable for both ears by means of a simple transfer of the retaining bar 12.

The positioning of the end portion 11A is carried out such that the radiation, which exists in a non focused manner in an approximately taper-shape enters the auditory canal and hits on the ear drum with a power of about 3-4 mW.

As an exact localization of the damage ear cells, by the stimulation of which one hopes to achieve an amelioration of the pain, is not possible, the described simple construction of the ear holder 10 is an optimal compromise between technical complexity and medical effect.

Avoiding a focusing at the exit from the end portion 11A ensures avoiding damages to the cells, which could occur if focusing regions having a high power density come to lie on points of the auditory canal or the ear drum.

For introducing the laser radiation of the laser 40 into the optical wave guide 20, a second transition piece 30 is provided as focusing sleeve, having a conical inner peripheral surface tapering in the direction of the entrance opening of the optical wave guide 20 and which is dimensioned such that the focusing region F of the laser light is immediately ahead of the entrance opening of the optical wave guide 20. The inner surface is coated, for example with chromium, gold or nickel, for enhancing reflection.

The second transition piece 30 is provided with a cylindrical bushing at its end portion on the side of the optical wave guide, in which a pressing tip 32 is introduced which accommodates the end portion of the optical wave guide 20 on the laser side. The pressing tip 32 comprises a through hole 33 with a first portion 33A with a first radius R1 for this purpose, into which the end portion of the coated optical wave guide 20 is glued in, as well as a continuing second portion 33B (approximately 5 mm in length) having a second radius R2, into which a de-coated end portion 20A of the optical wave guide protrudes by its entire length. The pressing tip 32 is inserted into the sleeve of the transition piece 30, for example by force fitting, such that the entrance cross section of the end of the optical wave guide 20A is immediately behind the focusing region F of the laser radiation and, accordingly, a substantially loss free introduction of the laser light into the optical wave guide 20 is ensured.

The second transition piece 30 and the laser 40 are connected by means of a connecting portion 42, for example in the type of a bayonet catching, as it is implied in FIG. 5, or for example by means of screwing.

The laser comprises at its outlet end a tapered holding section 41, onto which a ring 39 is slid, which comprises holders 34, 35 on both sides for restoring a holding member 50, for example by means of eyelet members 54, 55, such that the laser 40 can be carried by the patient to further reduce the moment acting on the transition piece 11.

This construction ensures a very easy handling, combined with a safe and individually adjustable seat of the ear holder, and, accordingly, a reliable radiation onto the desired portion of the inner ear.

1. Transmission device for introducing optical radiation into the ear, comprising an optical wave guide which connects a light source to an ear holder to output said radiation, characterized in that the ear holder (10) comprises a first transition piece (11), wherein the end area of the optical wave guide (20) is maintained, and a retaining bar (12), which can be introduced into the transition piece and can be bent in such a manner that the outlet (22) of the optical wave guide (20) can be positioned on the ear.

2. Transmission device according to claim 1, characterized in that the first transition piece is embodied in an elbow-shaped manner.

3. Transmission device according to claim 2, characterized in that the transition piece (11) comprises two channel shaped half shells (11.2, 11.2), of which at least one comprises wedge-shaped ridges (11.2) in transverse direction which are pressed into the jacket of the optical wave guide (20) and which fix the optical wave guide (20).

4. Transmission device according to claim 1, characterized in that the end area (11A) directed to the ear of the first transition piece 11 is enclosed by a replaceable ear piece (13).

5. Transmission device according to claim 1, characterized in that a second transition piece is provided (30) for introducing the laser radiation into the optical wave guide (20), which serves at least partially as focusing sleeve for the laser radiation and has a conical tapered inner shape, and in the end area (31) of which, which is shaped in the form of a hollow cylinder, the optical wave guide (20) is positioned, wherein the focusing region (F) generated in the focusing sleeve of the laser radiation is immediately ahead of the entrance opening of the optical wave guide (20).

6. Transmission device according to claim 5, characterized in that a pressing tip (32) is fixed to the hollow cylindrical end portion (31), accommodating the end portion of the optical wave guide (20) at the laser side.

7. Transmission device according to claim 5, characterized in that the cone-shaped inner surface of the second transmission piece (30) is metallized at least in its conical portion.

8. Transmission device according to claim 6, characterized in that the pressing tip (32) comprises a through bore (33) having a first portion (33A) with a radius (R1), into which the end portion of the coated optical wave guide (20) is inserted, in particular glued, and a second continuing portion (33B), having a second radius (R2), into which a de-coated end portion (20A) of the optical wave guide (20) is protruding with its entire length.

9. Transmission device according to claim 5, characterized in that the second transition piece (30) comprises at least one holder (34, 35) for a bearing member for the laser (40).

10. Transmission device according to claim 9, characterized in that the second transition piece (30) and the laser (40) are detachably connectable by means of a coupling portion (42).

11. Transmission device according to claim 9, characterized in that the holders (34, 35) are disposed at a ring (39), which seats on a tapered holding portion (41) of the laser (40).
